(19) United States
(12) Patent Application Publication
(10) Pub. No.: US 2025/0172563 A1
(43) Pub. Date: May 29, 2025

(54) MATERIALS AND METHODS FOR THE DIAGNOSIS AND TREATMENT OF CANCER

(71) Applicant: UNIVERSITY OF FLORIDA RESEARCH FOUNDATION, INCORPORATED, Gainesville, FL (US)

(72) Inventors: Sergei Alekseyevich Kusmartsev, Gainesville, FL (US); Paul Crispen, Gainesville, FL (US); Paul R. Dominguez-Gutierrez, Gainesville, FL (US); William L. Donelan, Gainesville, FL (US)

(21) Appl. No.: 18/912,748

(22) Filed: Oct. 11, 2024

Related U.S. Application Data
(63) Continuation of application No. 18/008,535, filed on Dec. 7, 2022.
(60) Provisional application No. 63/156,993, filed on Mar. 5, 2021.
(60) Provisional application No. 63/037,855, filed on Jun. 11, 2020.
(60) Provisional application No. 63/036,206, filed on Jun. 8, 2020.

(51) Int. Cl. G01N 33/574; A61K 39/00

(52) U.S. Cl. CPC G01N 33/57492; G01N 33/57407; A61K 2039/5152; A61K 2039/5154; G01N 2333/924

(57) ABSTRACT

The present disclosure provides a method of diagnosing cancer in a subject comprising contacting a blood sample from the subject with an hyaluronidase 2-binding agent and detecting a level of hyaluronidase 2-positive myeloid cells in the blood sample, wherein an elevated level of hyaluronidase 2-positive myeloid cells in the blood sample identifies the subject as suffering from cancer. Methods comprising administering a cancer therapy to a subject identified as suffering from cancer are also provided.

### CROSS-REFERENCE TO RELATED APPLICATIONS

The present application is a continuation application of U.S. application Ser. No. 18/008,535, filed Dec. 7, 2022, which is a national phase application of PCT/US21/36449, filed Jun. 8, 2021, which claims the benefit of priority to U.S. Provisional Application Nos. 63/036,206, filed Jun. 8, 2020, 63/037,855, filed Jun. 11, 2020, and 63/156,993 filed Mar. 5, 2021, the disclosure of which are incorporated herein by reference in their entireties.

### FIELD OF THE INVENTION

The present invention concerns the field of cancer diagnostics and therapeutics.

### INCORPORATION BY REFERENCE

This application contains, as a separate part of the disclosure, a sequence listing in computer-readable form (filename: 54895_SeqListing.txt, 8,533 bytes, created Jun. 8, 2021), which is incorporated by reference in its entirety.

### BACKGROUND

The tumor stroma, which is comprised of cellular and extracellular components, plays a major role in tumor growth and progression. The extracellular matrix (ECM) of tumors includes proteoglycans and glycans such as hyaluronic acid, also called hyaluronan (HA). HA is a member of the glycosaminoglycan family of polysaccharides synthesized at the cell surface and is characterized by very high molecular weights (2×10⁵ to 10×10⁶ kDa) and extended lengths of 2-25 μm (1). Increased HA synthesis is associated with wound healing and tumor growth (1-2). Several cancer types including breast, prostate, brain, lung and bladder are highly enriched with HA (2-4). Within the tumor tissue, HA buildup is frequently associated with increased degradation of HA, leading to accumulation of low molecular weight HA (LMW-HA) fragments (5, 6). Several studies have demonstrated that LMW-HA displays unique biologic activities that are not shared by high molecular weight HA (HMW-HA) (7-8). HMW-HA is anti-oncogenic, anti-inflammatory and anti-angiogenic, while LMW-HA promotes inflammation and tumor angiogenesis by stimulating expression of cytokines, chemokines and growth factors in TLR2/TLR4 dependent manner (9). In addition, LMW-HA is a potent inducer of cPLA2 activity in macrophages that promotes release of arachidonic acid; a substrate for inflammation-associated lipid mediators PGE2 and leukotrienes (9).

Myeloid cells, including tumor associated myeloid cells (TAMs) and myeloid-derived suppressor cells (MDSCs) represent a major cellular component in tumor tissues that play a key role in tumor development and progression (20-22). Recruitment of myeloid cells to the tumor microenvironment is a constant process fueled by the increased secretion of chemokines by both malignant as well as by stromal cells, which causes the mobilization of bone-marrow derived myeloid cell precursors from bone marrow and extravasation from the circulation into the tumor. Due to a tolerogenic cytokine milieu in the tumor microenvironment, recruited myeloid cells differentiate into immunosuppressive TAMs and MDSCs. Tumor-recruited myeloid cells have been shown to exert supportive tumor-promoting effects via multiple pathways that stimulate local immune suppression/tolerance, tumor angiogenesis, tissue remodeling and cancer inflammation. However, their role in the degradation of extracellular HA and, particularly, of tumor-associated HA has not been recognized yet.

The progression of bladder cancer is associated with enhanced expression of hyaluronidase 2 (Hyal2) RNA in tumor tissue (10, 11). Hyal2, a member of hyaluronidase family, is a glycosylphosphatidylinositol-linked (GPI-linked) enzyme that is anchored to the plasma membrane and is involved in the degradation of extracellular HA (12, 13). Hyal2 cleaves high molecular weight HA into intermediate size 20 kDa fragments. In addition to increased Hyal2 expression, bladder cancer tissue is frequently infiltrated with inflammatory and immune cells (14, 15).

### SUMMARY

In one aspect, described herein is a method of diagnosing cancer in a subject comprising contacting a blood sample from the subject with an hyaluronidase 2-binding agent and detecting a level of hyaluronidase 2-positive myeloid cells in the blood sample, wherein an elevated level of hyaluronidase 2-positive myeloid cells in the blood sample identifies the subject as suffering from cancer. In some embodiments, the cancer is prostate cancer, bladder cancer, breast cancer, brain cancer or lung cancer.

In another aspect, described herein is a method of treating cancer in a subject in need thereof, comprising detecting an elevated level of hyaluronidase 2-myeloid cells in a blood sample from the subject and administering a cancer therapy to the subject.

In yet another aspect, described herein is a method of monitoring efficacy of treatment in a subject undergoing treatment of bladder cancer, the method comprising detecting a level of hyaluronidase 2-positive myeloid cells in a blood sample from the subject before and after treatment with a cancer therapy, wherein a decreased level of hyaluronidase 2-positive myeloid cells identified in the blood sample after cancer therapy compared to before treatment began is indicative of effective treatment. A decreased level of hyaluronidase 2-positive myeloid cells (such that the level of hyaluronidase 2-positive myeloid cells in the sample is commensurate with a level of hyaluronidase 2-positive myeloid cells in healthy subjects) in the sample is indicative of effective cancer therapy.

The steps of determining the level of hyaluronidase 2-positive myeloid cells in the sample is performed by any means, such as those known in the art and described below. For example, the determining the level of hyaluronidase 2-positive myeloid cells in a sample optionally entails quantifying the amount of hyaluronidase-2 protein in the sample. In some embodiments, the method comprises isolating a population of myeloid cells from the sample, separating hyaluronidase-2 positive myeloid cells from the population of myeloid cells and quantifying the amount of hyaluronidase 2-positive myeloid cells in the sample.

In some embodiments, the blood sample is a peripheral blood sample.

In some embodiments, the myeloid cell is a CD11b+ cell, and optionally further expresses HLA-DR. In some embodiments, the cell does not express CD14.

In any of the methods described herein, the Hyal2 binding agent, in some embodiments, is an antibody. In some embodiments, the antibody comprises a light chain variable region set forth in SEQ ID NO: 2 and/or a heavy chain variable region set forth in SEQ ID NO: 4.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. With respect to aspects of the invention described or claimed with “a” or “an,” it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.

### BRIEF DESCRIPTION OF THE FIGURES

FIGS. 1A and 1B are graphs showing results of analysis of tumor-produced hyaluron using cytokines/chemokine antibody assays.

FIGS. 2A and 2B demonstrate enhanced HA degradation, accumulation of low molecular weight hyaluronan (LMW-HA) and elevated production of cytokines/chemokines in human bladder cancer tissue. Precision-cut tissue slices were prepared from freshly obtained normal and tumor human bladder tissue pieces and cultured in 24-well plates in full culture medium. Cell-free supernatants were collected on days 5-7, stored at 800° C. until analysis of tumor-produced HA using polyacrylamide gel electrophoresis (FIG. 2A) and cytokines/chemokine antibody arrays (FIG. 2B).

FIGS. 3A-3B show the identification of Hyal2-expressing myeloid cell subsets in peripheral blood from bladder cancer patients. FIG. 3A: Up-regulated expression of Hyal2 by peripheral blood-derived CD11b myeloid cells from cancer patients. CD11b myeloid cells were isolated from the peripheral blood of normal individuals or cancer patients using magnetic beads, stained with anti-Hyal2-PE antibodies and analyzed by IF microscopy. Percent of Hyal2+ cells was evaluated using immunofluorescent imaging microscope. Average means±SD are shown; *, P<0.05. FIG. 3B: Analysis of Hyal2 expression in blood-derived myeloid cells using flow cytometry. CD11b myeloid cells were isolated from the peripheral blood of cancer patients and cultured in complete culture medium for 48 hours in the presence or absence of TCM. T24 tumor cell-derived culture supernatant was a source of TCM in these experiments. Collected cells washed with PBS and stained with anti-Hyal2 Abs (FIG. 3B). Expression of indicated markers was measured using flow cytometry.

FIG. 4 is a graph showing the quantification of HA fragments in cytokine-treated Hyal2+ cells.

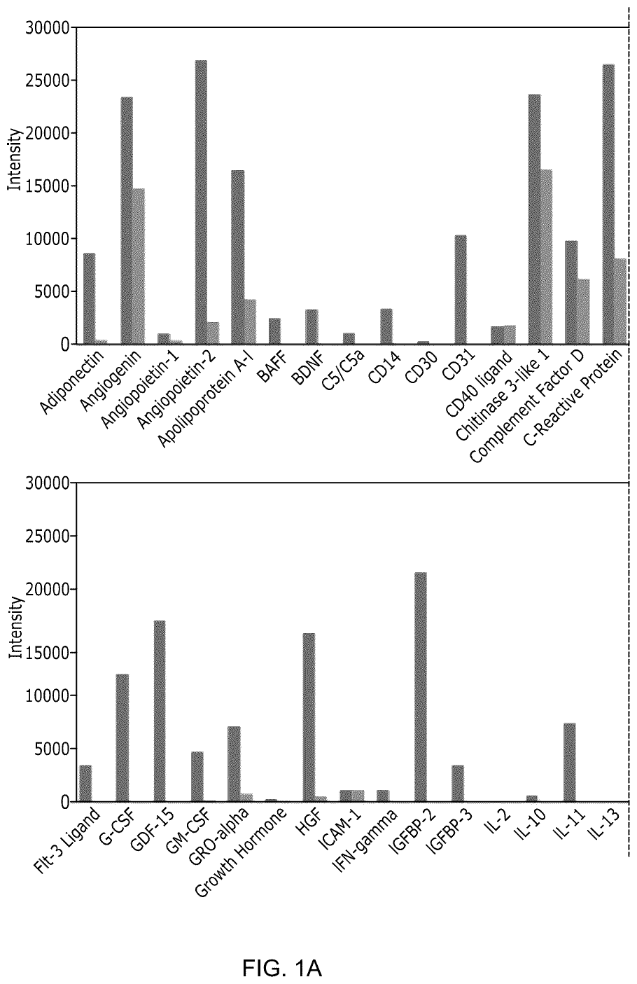

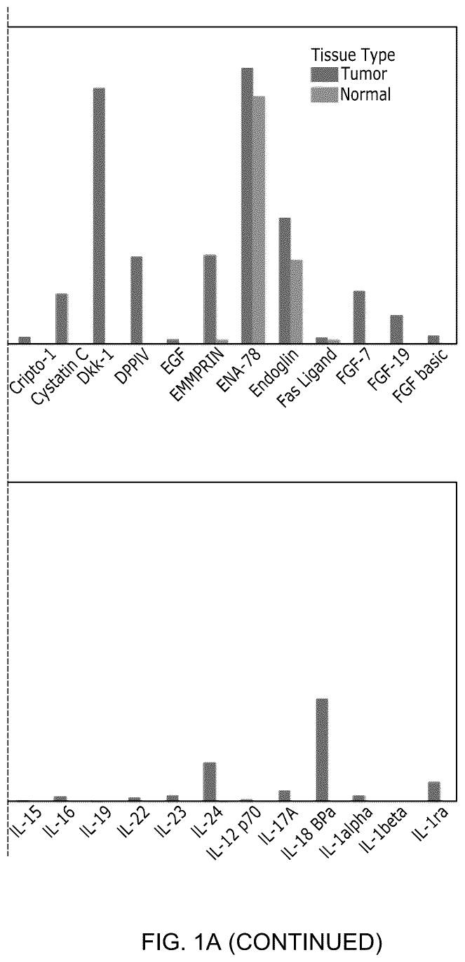

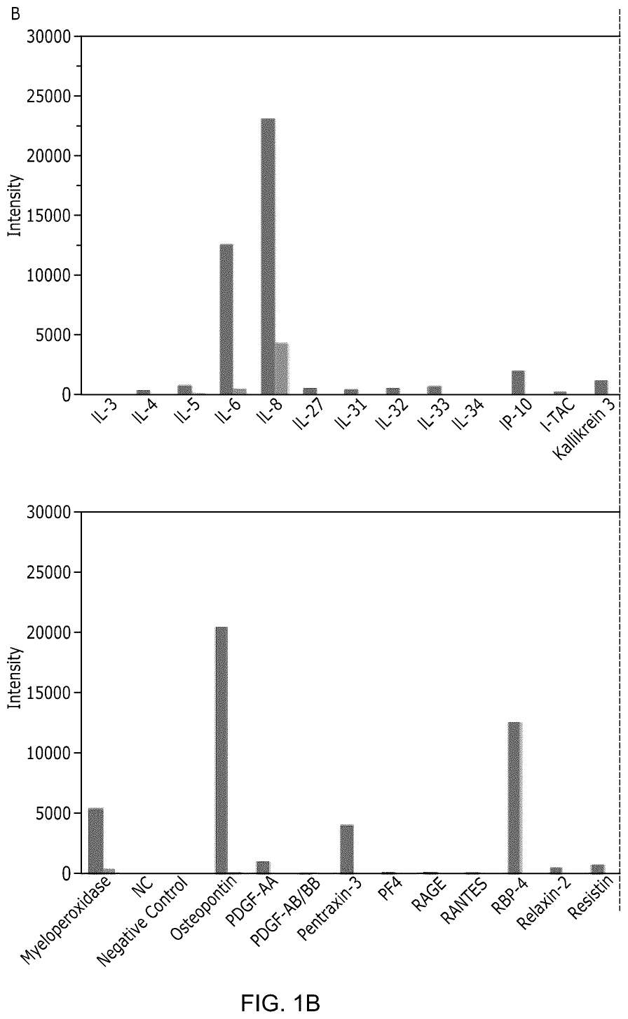

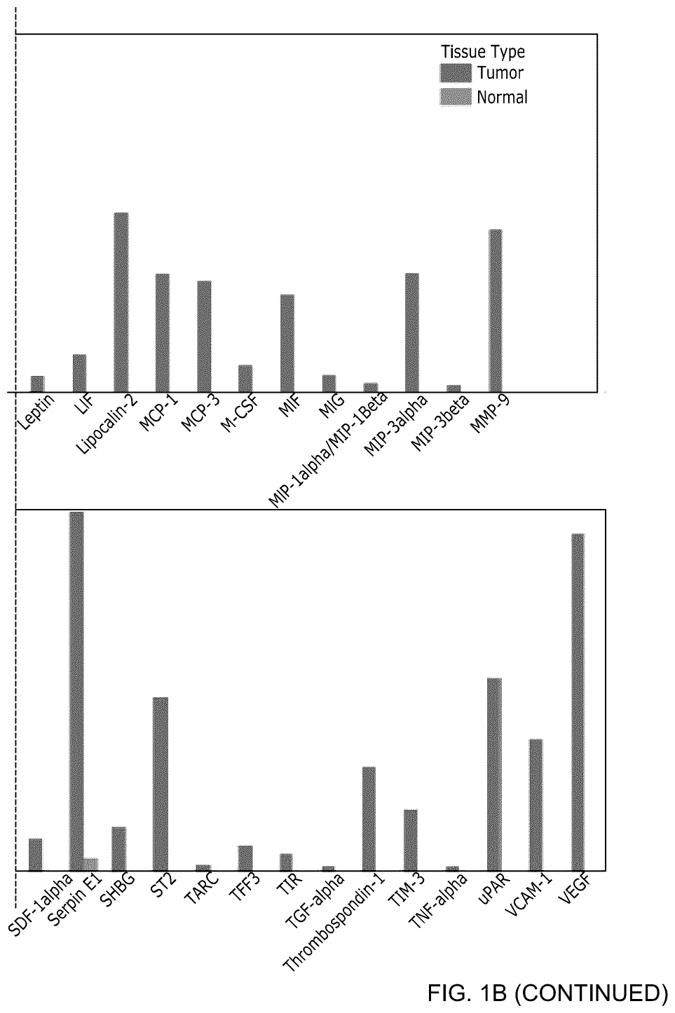

### DETAILED DESCRIPTION

The present disclosure is based on the discovery that increased circulating levels of hyaluronidase 2-positive myeloid cells is indicative of cancer in a subject. Data provided herein demonstrates that an increased presence of hyaluronidase 2 (hyal2)-positive myeloid cells was detected in the peripheral blood of subjects suffering from a cancer (including, but not limited to, bladder cancer, prostate cancer, breast cancer, brain cancer and lung cancer). This finding offers a new clinical insight into the diagnosis of certain cancers without the need for an invasive tissue biopsy.

### Myeloid Cells

Myeloid derived suppressor cells (MDSCs) are a population of early myeloid cells that are expanded in various disease states including cancer and are capable of suppressing the immune response (Gabrilovich et al., Nat. Rev. Immunol., 9:162-174, 2009 and Greten et al., Int. Immunopharmacol., 11:802-807, 2011, the disclosures of which are incorporated herein by reference in their entireties). In mice, MDSCs express myeloid markers (Gr1 or CD11b). In humans, the Gr1 antigen is absent. Human MDSCs express myeloid cell markers such as CD11b⁺ and CD33⁺, but are usually negative for lineage specific antigens such as CD3, CD19 and CD57. Monocytic MDSCs are usually characterized by HLA-DR^{-/low}, CD11b⁺, CD33⁺ and CD14⁺ phenotype in humans (CD11b⁺, Ly6G⁻/Ly6G⁺ in mice) whereas mature monocytes express high levels of HLA-DR. Granulocytic MDSCs are usually characterized by HLA-DR^{-/low}, CD11b⁺, CD33⁺, CD15⁺ phenotype in humans (CD11b⁺, Ly6G⁻/Ly6G^{low} in mice). MDSCs, monocytic MDSCs and granulocytic MDSCs have all been shown to possess immunosuppressive properties (Filipazzi et al., Cancer Immunol. Immunother., 61:255-263, 2012; Mundy-Bosse et al., Cancer Immunol. Immunother., 60:1269-1271, 2011; Movahedi et al., Blood, 111:4233-4244, 2008, the disclosure of which are incorporated herein by reference in their entireties).

MDSCs can be generated in the bone marrow in response to cancer derived factors such as granulocyte colony stimulating factor (G-CSF), IL-6, granulocyte monocyte colony stimulating factor (GM-CSF), IL1β, prostaglandin E2 (PGE2), tumor necrosis factor α and vascular endothelial growth factor (VEGF) and are recruited to the tumor site by CCL2, CXCL12 and CXCL5. Additional signals stimulate MDSCs to acquire immunosuppressive properties which are mediated through members of the signal transducer and activator of transcription (STAT1, STAT3, STAT6) and nuclear factor kappa light chain enhancer of activated B cells (NFκB) transcription factors (Gabrilovich, supra). Activated MDSCs produce Arginase 1 (ARG1), inducible nitric oxide synthase (NOS2), IDO (indoleamine 2,3-dioxygenase), NADPH oxidase and immunosuppressive cytokines that have the potential to inhibit cytotoxic T lymphocytes (CTLs), dendritic cells and natural killer cells as well as expand CD4⁺CD25⁺FoxP3⁺ regulatory T cells (Tregs).

In one aspect, described herein is a method of diagnosing cancer in a subject comprising contacting a blood sample from the subject with an hyaluronidase 2-binding agent and detecting a level of hyaluronidase 2-positive myeloid cells in the blood sample, wherein an elevated level of hyaluronidase 2-positive myeloid cells in the blood sample identifies the subject as suffering from cancer. In some embodiments, the cancer is prostate cancer, bladder cancer, breast cancer, brain cancer or lung cancer.

The methods may further comprise the step of comparing the level of hyaluronidase 2-positive myeloid cells in a blood sample to a predetermined criterion. In related embodiments, the method of diagnosing comprises detecting a level of hyaluronidase 2-positive myeloid cells that falls within a predetermined range indicative of cancer. This predetermined range of levels is typically different from (higher than) the levels of circulating hyaluronidase 2-positive myeloid cells in subjects without cancer.

The term “predetermined criterion” as used herein refers to a number indicative of the level of circulating hyaluronidase 2-positive myeloid cells obtained from prior measurements of hyaluronidase 2-positive myeloid cells in blood samples from a plurality of subjects without cancer. In some variations, the predetermined criterion is the level of hyaluronidase 2-positive cells in healthy human controls (i.e., subjects with no clinical manifestation of cancer), in which case the level of hyaluronidase 2-positive myeloid cells determined in the method disclosed herein is increased compared to the level of hyaluronidase 2-positive myeloid cells in a blood sample obtained from the healthy controls. In some embodiments, the level of hyaluronidase 2-positive myeloid cells is increased by at least 8-fold (e.g., 8-fold, 9-fold, 10-fold, 11-fold, 12-fold, 13-fold, 14-fold, 15-fold, 20-fold or higher) compared to the level of hyaluronidase 2-positive myeloid cells in a blood sample obtained from the healthy controls.

The term “predetermined range” as used herein refers to a range of levels hyaluronidase 2-positive myeloid cells typically observed in human subjects with cancer (e.g., bladder cancer), in which case the level of hyaluronidase 2-positive cells is indicative of cancer if it falls within the predetermined range.

In other variations, the predetermined criterion or range might include information such as mean, standard deviation, quartile measurements, confidence intervals, or other information about the distribution or range of circulating hyaluronidase 2-positive myeloid cells in cancer subjects or subjects without cancer. In still other variations, the predetermined criterion is a receiver operating characteristic curve based on data of circulating hyaluronidase 2-positive myeloid cells in cancer subjects or subjects without cancer. In still other variations, the predetermined criterion is a cutoff value of hyaluronidase 2-positive myeloid cells, wherein the cutoff value is determined, based on previous measurements to discriminate cancer with a sensitivity and specificity calculated from measurements of circulating hyaluronidase 2-positive myeloid cells in human subjects with cancer and non-cancer human subjects. Optionally, the predetermined criterion is based on subjects further stratified by other characteristics that can be determined for a subject, to further refine the diagnostic precision. Such additional characteristics include, for example, sex, age, weight, smoking habits, race or ethnicity, blood pressure, other diseases, and medications.

To determine a measurement of hyaluronidase 2 in a blood sample, the sample is contacted with an anti-hyaluronidase 2 binding agent (e.g., antibody or antigen binding fragment thereof) for a time sufficient to allow immunocomplexes to form. Immunocomplexes formed between the antibody and hyaluronidase 2 in the blood sample are then detected. The amount of hyaluronidase 2 in the biological sample is optionally quantitated by measuring the amount of the immunocomplex formed between the antibody and the hyaluronidase 2. For example, the antibody can be quantitatively measured if it has a detectable label, or a secondary antibody can be used to quantify the immunocomplex.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats can readily be adapted to employ the antibodies (or fragments thereof) of the present disclosure. Examples of such assays can be found in Chard, T., An Introduction to Radioimmunoassay and Related Techniques, Elsevier Science Publishers, Amsterdam, The Netherlands (1986); Bullock, G.R. et al., Techniques in Immunocytochemistry, Academic Press, Orlando, FL Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985).

The assay described herein may be useful in, e.g., evaluating the efficacy of a particular therapeutic cancer treatment regime in animal studies, in clinical trials, or in monitoring the treatment of an individual patient.

In some embodiments, an anti-hyal2 antibody (or antigen binding fragment thereof) is attached to a solid support, and binding is detected by detecting a complex between the hyaluronidase 2 expressed on myeloid cells in the sample and the antibody (or antigen binding fragment thereof) on the solid support. The antibody (or fragment thereof) optionally comprises a detectable label and binding is detected by detecting the label in the anti-hyal2-antibody complex.

Detection of the presence or absence of a hyal2-antibody complex be achieved using any method known in the art. For cell free binding assays, one of the components usually includes, or is coupled to, a detectable label. A wide variety of labels can be used, such as those that provide direct detection (such as radioactivity, luminescence, optical or electron density) or indirect detection (such as epitope tag such as the FLAG epitope, enzyme tag such as horseradish peroxidase). The label can be bound to the antibody, or incorporated into the structure of the antibody. A variety of methods can be used to detect the label, depending on the nature of the label and other assay components. For example, the label can be detected while bound to the solid substrate or subsequent to separation from the solid substrate. Labels can be directly detected through optical or electron density, radioactive emissions, nonradiative energy transfers or indirectly detected with antibody conjugates, or streptavidin-biotin conjugates. Another example of a detection method is a reporter gene transcription assay, wherein a detectable product is produced upon binding of a hyal2 peptide interacting with an anti-hyal2 antibody (or other hyal2 binding agent). The detectable product may be observed by detecting e.g., β-galactosidase activity or luciferase activity.

To carry out the methods described herein, myeloid cells are harvested from a biological fluid sample. Biological fluids include, but are not limited to, blood, serum and plasma. The biological sample can be used (i) directly as obtained from the source or (ii) following a pre-treatment to modify the character of the sample. Thus, the sample can be treated prior to use in the disclosed methods by, for example, preparing plasma from blood, concentrating liquids, inactivating interfering components, removing heparin from the sample, adding reagents, and the like.

### Anti-Hyaluronidase 2 Binding Agents

In some embodiments, the anti-hyaluronidase 2 binding agent for use in the methods described herein is an anti-hyaluronidase 2 antibody. The term “antibody” refers to an intact immunoglobulin molecule (including polyclonal, monoclonal, chimeric, humanized, and/or human versions having full length heavy and/or light chains). The antibody may be any type of antibody, i.e., immunoglobulin, known in the art. In exemplary embodiments, the antibody is an antibody of class or isotype IgA, IgD, IgE, IgG, or IgM. In exemplary embodiments, the antibody described herein comprises one or more alpha, delta, epsilon, gamma, and/or mu heavy chains. In exemplary embodiments, the antibody described herein comprises one or more kappa or light chains. In exemplary aspects, the antibody is an IgG antibody and optionally is one of the four human subclasses: IgG1, IgG2, IgG3 and IgG4. Also, the antibody in some embodiments is a monoclonal antibody. In other embodiments, the antibody is a polyclonal antibody. In some aspects, the antibody is a chimeric or a humanized antibody. The term “humanized” when used in relation to antibodies refers to antibodies having at least CDR regions from a non-human source and which are engineered to have a structure and immunological function more similar to true human antibodies than the original source antibodies. For example, humanizing can involve grafting CDRs from a non-human antibody, such as a mouse antibody, into a human antibody framework. Humanizing also can involve select amino acid substitutions to make a non-human sequence look more like a human sequence.

“Specifically binds” as used herein means that the antibody (or antigen binding fragment) preferentially binds an antigen (e.g., human hyaluronidase 2 (Genbank Accession No. NP_003764) over other proteins. In some embodiments, “specifically binds” means the antibody has a higher affinity for the antigen than for other proteins. Antibodies that specifically bind an antigen may have a binding affinity for the antigen of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M. It will be appreciated that ranges having the values above as end points is contemplated in the context of the disclosure. For example, the antibody or antigen binding fragment thereof may bind human hyaluronidase 2 of SEQ ID NO: 5 with an affinity of about 1×10⁻⁷ M to about 9×10⁻¹² M or an affinity of 1×10⁻⁹ to about 9×10⁻¹².

In some or any embodiments, the antibody (or antigen binding fragment) binds to human hyaluronidase 2 of SEQ ID NO: 5, or a naturally occurring variant thereof, with an affinity (Kd) of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M, or less than or equal to 1×10⁻¹² M, or ranging from 1×10⁻⁹ to 1'10⁻¹⁰, or ranging from 1×10⁻¹² to about 1×10⁻¹³. Affinity is determined using a variety of techniques, examples of which include an affinity ELISA assay and a surface plasmon resonance (BIAcore) assay.

“CDR” refers to the complementarity determining region within antibody variable sequences. There are three CDRs in each of the variable regions of the heavy chain and the light chain, which are designated CDR1, CDR2 and CDR3, for each of the variable regions. The term “set of six CDRs” as used herein refers to a group of three CDRs that occur in the light chain variable region and heavy chain variable region, which are capable of binding the antigen. The exact boundaries of CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively.

In various aspects, the antibody (or antigen binding fragment thereof) comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 6, CDR-H2 has the sequence given in SEQ ID NO: 7, CDR-H3 has the sequence given in SEQ ID NO: 8, CDR-L1 has the sequence given in SEQ ID NO: 9, CDR-L2 has the sequence given in SEQ ID NO: 10 and CDR-L3 has the sequence given in SEQ ID NO: 11. In various aspects, the antibody (or antigen binding fragment thereof) comprises a CDR-H1 having the sequence given in SEQ ID NO: 6 with 3, 2, or 1 amino acid substitutions therein; CDR-H2 having the sequence given in SEQ ID NO: 7 with 3, 2, or 1 amino acid substitutions therein; CDR-H3 having the sequence given in SEQ ID NO: 8 with 3, 2, or 1 amino acid substitutions therein; CDR-L1 having the sequence given in SEQ ID NO: 9 with 3, 2, or 1 amino acid substitutions therein; CDR-L2 having the sequence given in SEQ ID NO: 10 with 3, 2, or 1 amino acid substitutions therein and CDR-L3 having the sequence given in SEQ ID NO: 7 with 3, 2, or 1 amino acid substitutions therein. The anti-hyaluronidase 2 antibody, in various aspects, comprises two of the CDRs, three of the CDRs, four of the CDRs, five of the CDRs or all six of the CDRs. In some embodiments, the anti-hyaluronidase 2 antibody comprises a set of six CDRs as follows: CDR-H1 of SEQ ID NO: 7, CDR-H2 of SEQ ID NO: 7, CDR-H3 of SEQ ID NO: 8, CDR-LI of SEQ ID NO: 9, CDR-L2 of SEQ ID NO: 10 and CDR-L3 of SEQ ID NO: 11.

In some or any embodiments, the antibody (or fragment thereof) comprises a light chain variable region comprising an amino acid sequence having at least 75% identity (e.g., at least 75%, 80%, 85%, 90%, 95% or 100% identity) to the amino acid sequence set forth in SEQ ID NO: 2 and/or a heavy chain variable region comprising an amino acid sequence having at least 75% identity (e.g., at least 75%, 80%, 85%, 90%, 95% or 100% identity) to the amino acid sequence set forth in SEQ ID NO: 4. In various aspects, the difference in the sequence compared to SEQ ID NO: 2 or SEQ ID NO: 4 lies outside the CDR region in the corresponding sequences.

In some or any embodiments, the antibody (or antigen binding fragment) comprises a light chain variable region comprising an amino acid sequence set forth in SEQ ID NO: 2 and a heavy chain variable region comprising an amino acid sequence set forth in SEQ ID NO: 4.

Antigen binding fragments of anti-hyaluronidase 2 antibodies described herein are also contemplated. The antigen binding fragment can be any part of an antibody that has at least one antigen binding site, and the antigen binding fragment may be part of a larger structure (an “antibody product”) that retains the ability of the antigen binding fragment to recognize hyaluronidase 2. For ease of reference, these antibody products that include antigen binding fragments are included in the disclosure herein of “antigen binding fragment.” Examples of antigen binding fragments, include, but are not limited to, Fab, F(ab′)₂, a monospecific or bispecific Fab₂, a trispecific Fab₃, scFv, dsFv, scFv-Fc, bispecific diabodies, trispecific triabodies, minibodies, a fragment of IgNAR (e.g., V-NAR), a fragment of hcIgG (e.g., VhH), bis-scFvs, fragments expressed by a Fab expression library, and the like. In exemplary aspects, the antigen binding fragment is a domain antibody, VhH domain, V-NAR domain, VH domain, VL domain, or the like. Antibody fragments of the disclosure, however, are not limited to these exemplary types of antibody fragments. In exemplary aspects, antigen binding fragment is a Fab fragment. In exemplary aspects, the antigen binding fragment comprises two Fab fragments. In exemplary aspects, the antigen binding fragment comprises two Fab fragments connected via a linker. In exemplary aspects, the antigen binding fragment comprises or is a minibody comprising two Fab fragments.

A domain antibody comprises a functional binding unit of an antibody, and can correspond to the variable regions of either the heavy (V_{H}) or light (V_{L}) chains of antibodies. A domain antibody can have a molecular weight of approximately 13 kDa, or approximately one-tenth of a full antibody. Domain antibodies may be derived from full antibodies such as those described herein.

In some embodiments, the scFv is attached to a human Fc domain. In some embodiments, the Fc domain does not activate Fc effector functions.

Suitable methods of making antibodies are known in the art. For instance, standard hybridoma methods are described in, e.g., Harlow and Lane (eds.), Antibodies: A Laboratory Manual, CSH Press (1988), and CA. Janeway et al. (eds.), Immunobiology, 5th Ed., Garland Publishing, New York, NY (2001)). Monoclonal antibodies for use in the methods of the disclosure may be prepared using any technique which provides for the production of antibody molecules by continuous cell lines in culture. These include but are not limited to the hybridoma technique originally described by Koehler and Milstein (Nature 256:495-497, 1975), the human B-cell hybridoma technique (Kosbor et al., Immunol Today 4:72, 1983; Cote et al., Proc Natl Acad Sci 80:2026-2030, 1983) and the EBV-hybridoma technique (Cole et al., Monoclonal Antibodies and Cancer Therapy, Alan R Liss Inc, New York N.Y., pp 77-96, (1985). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. No. 5,545,806, 5,569,825, and 5,714,352, and U.S. Patent Application Publication No. 2002/0197266 A1). Antibodies may also be produced by inducing in vivo production in the lymphocyte population or by screening recombinant immunoglobulin libraries or panels of highly specific binding reagents as disclosed in Orlandi et al (Proc Natl Acad Sci 86:3833-3837; 1989), and Winter G and Milstein C (Nature 349:293-299, 1991). If the full sequence of the antibody or antigen-binding fragment is known, then methods of producing recombinant proteins may be employed. See, e.g., “Protein production and purification” Nat Methods 5(2): 135-146 (2008). In some embodiments, the antibodies (or antigen binding fragments) are isolated from cell culture or a biological sample if generated in vivo.

Phage display also can be used to generate the antibody of the present disclosures. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3rd Edition, Cold Spring Harbor Laboratory Press, New York (2001)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150). Related methods also are described in U.S. Pat. Nos. 5,403,484; 5,571,698; 5,837,500; 5,702,892. The techniques described in U.S. Pat. Nos. 5,780,279; 5,821,047; 5,824,520; 5,855,885; 5,858,657; 5,871,907; 5,969,108; 6,057,098; and 6,225,447.

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods of testing antibodies for the ability to bind to an epitope of hyaluronidase 2, regardless of how the antibodies are produced, are known in the art and include, e.g., radioimmunoassay (RIA), ELISA, Western blot, immunoprecipitation, surface plasmon resonance (e.g., BIAcore), and competitive inhibition assays (see, e.g., Janeway et al., infra, and U.S. Patent Application Publication No. 2002/0197266).

Antibody fragments that contain the antigen binding, or idiotype, of the antibody molecule may be generated by techniques known in the art. For example, a F(ab′)₂ fragment may be produced by pepsin digestion of the antibody molecule; Fab′ fragments may be generated by reducing the disulfide bridges of the F(ab′)₂ fragment; and two Fab' fragments which may be generated by treating the antibody molecule with papain and a reducing agent. The disclosure is not limited to enzymatic methods of generating antigen binding fragments; the antigen binding fragment may be a recombinant antigen binding fragment produced by expressing a polynucleotide encoding the fragment in a suitable host cell.

A single-chain variable region fragments (scFv), which consists of a truncated Fab fragment comprising the variable (V) domain of an antibody heavy chain linked to a V domain of an antibody light chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7, 697-704 (1994)).

Recombinant antibody fragments, e.g., scFvs, can also be engineered to assemble into stable multimeric oligomers of high binding avidity and specificity to different target antigens. Such diabodies (dimers), triabodies (trimers) or tetrabodies (tetramers) are well known in the art, see e.g., Kortt et al., Biomol Eng. 2001 18:95-108, (2001) and Todorovska et al., J Immunol Methods. 248:47-66, (2001).

### Antibody-Drug Conjugates

The antibody of the disclosure is optionally conjugated with drugs to form antibody-drug conjugates (ADCs). In general, ADCs are used in a variety of contexts, including oncology applications, where the use of antibody-drug conjugates for the local delivery of cytotoxic or cytostatic agents allows for the targeted delivery of the drug moiety to tumors, which can allow higher efficacy, lower toxicity, etc. An overview of this technology is provided in Ducry et al., Bioconjugate Chem., 21:5-13 (2010); Carter et al., Cancer J. 14(3):154 (2008); and Senter, Current Opin. Chem. Biol. 13:235-244 (2009), all of which are hereby incorporated by reference in their entirety.

Generally, conjugation is performed by covalent attachment to an antibody, as further described below, and generally relies on a linker, often a peptide linkage (which, as described herein, may be designed to be sensitive to cleavage by proteases at the target site or not). In addition, linkage of the linker-drug unit (LU-D) can be achieved by attachment to cysteines within the antibody. The number of drug moieties per antibody can change, depending on the conditions of the reaction, and can vary from 1:1 to 10:1 drug: antibody. As will be appreciated by those in the art, the actual number is an average.

The drug of the ADC can be selected from any of a number of agents, including but not limited to cytotoxic agents such as chemotherapeutic agents, growth inhibitory agents, toxins (for example, an enzymatically active toxin of bacterial, fungal, plant, or animal origin, or fragments thereof), or a radioactive isotope (that is, a radioconjugate). The disclosure further provides methods of using the ADCs.

Drugs for use in the context of the disclosure include cytotoxic drugs, particularly those which are used for cancer therapy. Such drugs include, in general, DNA damaging agents, anti-metabolites, natural products and their analogs. Exemplary classes of cytotoxic agents include the enzyme inhibitors such as dihydrofolate reductase inhibitors, and thymidylate synthase inhibitors, DNA intercalators, DNA cleavers, topoisomerase inhibitors, the anthracycline family of drugs, vinca drugs, mitomycins, bleomycins, cytotoxic nucleosides, the pteridine family of drugs, diynenes, podophyllotoxins, dolastatins, maytansinoids, differentiation inducers, and taxols.

In some embodiments, an antibody described herein is conjugated with methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin and podophyllotoxin derivatives such as etoposide or etoposide phosphate, vinblastine, vincristine, vindesine, taxanes including taxol, taxotere retinoic acid, butyric acid, N8-acetyl spermidine, camptothecin, calicheamicin, esperamicin, ene-diynes, duocarmycin A, duocarmycin SA, calicheamicin, camptothecin, maytansinoids (including DM1), monomethylauristatin E (MMAE), monomethylauristatin F (MMAF), or maytansinoids (DM4) and their analogues.

In some embodiments, an antibody described herein is conjugated with Doxorubicin, Docetaxel (Taxotere), Cisplatin, Gemcitabine, Carboplatin, Etoposide, Cyclophosphamide, Epirubicin, Vincristine, Methotrexate, 5-fluoroucil, or Mitomycin C.

### Therapeutic Methods

In another aspect, described herein is a method of treating a cancer associated with elevated circulating levels hyaluronidase 2-positive myeloid cells, the method comprising detecting an elevated level of hyaluronidase 2-myeloid cells in a blood sample from the subject as described herein and administering a cancer therapy to the subject.

Exemplary cancers include, but are not limited to, breast cancer, renal cell carcinoma, esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIIA skin melanoma, stage IIIB skin melanoma, stage IIIC skin melanoma, stage IV skin melanoma, malignant melanoma of head and neck, lung cancer, non-small cell lung cancer (NSCLC), squamous cell non-small cell lung cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission, adult acute myeloid leukemia with Inv(16)(p13.1q22); CBFB-MYH11, adult acute myeloid leukemia with t(16; 16)(p13.1; q22); CBFB-MYH11, adult acute myeloid leukemia with t(8; 21)(q22; q22); RUNX1-RUNX1T1, adult acute myeloid leukemia with t(9; 11)(p22; q23); MLLT3-MLL, adult acute promyelocytic leukemia with t(15; 17)(q22; q12); PML-RARA; alkylating agent-related acute myeloid leukemia, chronic lymphocytic leukemia, richter's syndrome, waldenstrom macroglobulinemia, adult glioblastoma, adult gliosarcoma, recurrent glioblastoma, recurrent childhood rhabdomyosarcoma, recurrent Ewing sarcoma/peripheral primitive neuroectodermal tumor, recurrent neuroblastoma, recurrent osteosarcoma, colorectal cancer, MSI positive colorectal cancer, MSI negative colorectal cancer, nasopharyngeal nonkeratinizing carcinoma, recurrent nasopharyngeal undifferentiated carcinoma, cervical adenocarcinoma, cervical adenosquamous carcinoma, cervical squamous cell carcinoma, recurrent cervical carcinoma, stage IVA cervical cancer, stage IVB cervical cancer, anal canal squamous cell carcinoma, metastatic anal canal carcinoma, recurrent anal canal carcinoma, recurrent head and neck cancer, carcinoma, squamous cell of head and neck, head and neck squamous cell carcinoma (HNSCC), ovarian carcinoma, colon cancer, gastric cancer, advanced GI cancer, gastric adenocarcinoma, gastroesophageal junction adenocarcinoma, bone neoplasms, soft tissue sarcoma, bone sarcoma, thymic carcinoma, urothelial carcinoma, recurrent merkel cell carcinoma, stage III merkel cell carcinoma, stage IV merkel cell carcinoma, myelodysplastic syndrome and recurrent mycosis fungoides and Sezary syndrome.

In some embodiments, the cancer associated with elevated circulating levels hyaluronidase 2-positive myeloid cells is prostate cancer, bladder cancer, breast cancer, brain cancer or lung cancer. In some embodiments, the cancer is bladder cancer.

Exemplary cancer therapies include, but are not limited to, a surgical therapy, chemotherapy (e.g., administration of a protein kinase inhibitor or a EGFR-targeted therapy), radiation therapy, cryotherapy, hyperthermia treatment, phototherapy, radioablation therapy, hormonal therapy, immunotherapy, small molecule therapy, receptor kinase inhibitor therapy, anti-angiogenic therapy, cytokine therapy or a biological therapies such as monoclonal antibodies, siRNA, miRNA, antisense oligonucleotides, ribozymes or gene therapy. Without limitation the biological therapy may be a gene therapy, such as tumor suppressor gene therapy, a cell death protein gene therapy, a cell cycle regulator gene therapy, a cytokine gene therapy, a toxin gene therapy, an immunogene therapy, a suicide gene therapy, a prodrug gene therapy, an anti-cellular proliferation gene therapy, an enzyme gene therapy, or an anti-angiogenic factor gene therapy.

In some embodiments, the cancer therapy comprises chemotherapy. Exemplary chemotherapies include, but are not limited to, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above.

In some embodiments, the cancer therapy is an histone deacetylase inhibitor (e.g., gefitinib or Gleevac).

In some embodiments, the cancer therapy is radiotherapy. Other factors that cause DNA damage and have been used extensively include what are commonly known as y-rays, X-rays, and/or the directed delivery of radioisotopes to tumor cells. Other forms of DNA damaging factors are also known such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

In some embodiments, the cancer therapy is immunotherapy. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells as well as genetically engineered variants of these cell types modified to express chimeric antigen receptors.

Exemplary immunotherapies that can be combined with the agonist complexes described herein include immune adjuvants (e.g., Mycobacterium bovis, Plasmodium falciparum, dinitrochlorobenzene and aromatic compounds) (U.S. Pat. Nos. 5,801,005; 5,739,169; Hui and Hashimoto, 1998; Christodoulides et al., 1998), cytokine therapy (e.g., interferons .alpha., .beta. and .gamma.; interleukins (IL-1, IL-2), GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. Nos. 5,830,880 and 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the combination therapy described herein.

Other immunotherapies contemplated for use in methods of the present disclosure include those described by Tchekmedyian et al., 2015, incorporated herein by reference. The immunotherapy may comprise suppression of T regulatory cells (Tregs), myeloid derived suppressor cells (MDSCs) and cancer associated fibroblasts (CAFs). In some embodiments, the immunotherapy is a tumor vaccine (e.g., whole tumor cell vaccines, peptides, and recombinant tumor associated antigen vaccines), or adoptive cellular therapies (ACT) (e.g., T cells, natural killer cells, TILs, and LAK cells). The T cells may be engineered with chimeric antigen receptors (CARs) or T cell receptors (TCRs) to specific tumor antigens. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. In some aspects, the T cells are activated CD4 and/or CD8 T cells in the individual which are characterized by IFNγ “producing CD4 and/or CD8 T cells and/or enhanced cytolytic activity relative to prior to the administration of the combination. The CD4 and/or CD8 T cells may exhibit increased release of cytokines selected from the group consisting of IFN-γ, TNF-α and interleukins. The CD4 and/or CD8 T cells can be effector memory T cells. In certain embodiments, the CD4 and/or CD8 effector memory T cells are characterized by having the expression of CD44^{high} CD62^{low}.

Examples of monoclonal antibodies include, but are not limited to, trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-I131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and-2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex.TM. (17-1A) (murine monoclonal antibody); Panorex (@(17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Examples of antibodies include those disclosed in U.S. Pat. Nos. 5,736,167, 7,060,808, and 5,821,337.

Further examples of antibodies include anti-human OX40 agonist antibody (Genentech); Zanulimumab (anti-CD4 mAb), Keliximab (anti-CD4 mAb); Ipilimumab (MDX-101; anti-CTLA-4 mAb); Tremilimumab (anti-CTLA-4 mAb); (Daclizumab (anti-CD25/IL-2R mAb); Basiliximab (anti-CD25/IL-2R mAb); MDX-1106 (anti-PD1 mAb); antibody to GITR; GC1008 (anti-TGF-. beta. antibody); metelimumab/CAT-192 (anti-TGF-. beta.antibody); lerdelimumab/CAT-152 (anti-TGF-.beta.antibody); ID11 (anti-TGF-.beta. antibody); Denosumab (anti-RANKL mAb); BMS-663513 (humanized anti-4-1BB mAb); SGN-40 (humanized anti-CD40 mAb); CP870,893 (human anti-CD40 mAb); Infliximab (chimeric anti-TNF mAb; Adalimumab (human anti-TNF mAb); Certolizumab (humanized Fab anti-TNF); Golimumab (anti-TNF); Etanercept (Extracellular domain of TNFR fused to IgG1 Fc); Belatacept (Extracellular domain of CTLA-4 fused to Fc); Abatacept (Extracellular domain of CTLA-4 fused to Fc); Belimumab (anti-B Lymphocyte stimulator); Muromonab-CD3 (anti-CD3 mAb); Otelixizumab (anti-CD3 mAb); Teplizumab (anti-CD3 mAb); Tocilizumab (anti-IL6R mAb); REGN88 (anti-IL6R mAb); Ustekinumab (anti-IL-12/23 mAb); Briakinumab (anti-IL-12/23 mAb); Natalizumab (anti-.alpha.4 integrin); Vedolizumab (anti-.alpha.4.beta.7 integrin mAb); T1 h (anti-CD6 mAb); Epratuzumab (anti-CD22 mAb); Efalizumab (anti-CD11a mAb); and Atacicept (extracellular domain of transmembrane activator and calcium-modulating ligand interactor fused with Fc).

It is contemplated that other agents may be used in combination with the compositions provided herein to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the compositions provided herein by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the compositions provided herein to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the compositions provided herein to improve the treatment efficacy.

In some embodiments, cancer therapy comprises the use of one or more oncolytic viruses. Examples of oncolytic viruses include adenoviruses, adeno-associated viruses, retroviruses, lentiviruses, herpes viruses, pox viruses, vaccinia viruses, vesicular stomatitis viruses, polio viruses, Newcastle's Disease viruses, Epstein-Barr viruses, influenza viruses and reoviruses. In a particular embodiment, the other agent is talimogene laherparepvec (T-VEC) which is an oncolytic herpes simplex virus genetically engineered to express GM-CSF. Talimogene laherparepvec, HSV-1 [strain JS1] ICP34.5-/ICP47-/hGM-CSF, (previously known as OncoVEX.sup.GM CSF) is an intratumorally delivered oncolytic immunotherapy comprising an immune-enhanced HSV-1 that selectively replicates in solid tumors. (Lui et al., 2003; U.S. Pat. Nos. 7,223,593 and 7,537,924; incorporated herein by reference).

In some embodiments, hormonal therapy may also be used in conjunction with the in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

In some aspects, cancer therapy comprises the use of a protein kinase inhibitor or a monoclonal antibody that inhibits receptors involved in protein kinase or growth factor signaling pathways such as an EGFR, VEGFR, AKT, Erb1, Erb2, ErbB, Syk, Bcr-Abl, JAK, Src, GSK-3, PI3K, Ras, Raf, MAPK, MAPKK, mTOR, c-Kit, eph receptor or BRAF inhibitors. Nonlimiting examples of protein kinase or growth factor signaling pathways inhibitors include Afatinib, Axitinib, Bevacizumab, Bosutinib, Cetuximab, Crizotinib, Dasatinib, Erlotinib, Fostamatinib, Gefitinib, Imatinib, Lapatinib, Lenvatinib, Mubritinib, Nilotinib, Panitumumab, Pazopanib, Pegaptanib, Ranibizumab, Ruxolitinib, Saracatinib, Sorafenib, Sunitinib, Trastuzumab, Vandetanib, AP23451, Vemurafenib, MK-2206, GSK690693, A-443654, VQD-002, Miltefosine, Perifosine, CAL101, PX-866, LY294002, rapamycin, temsirolimus, everolimus, ridaforolimus, Alvocidib, Genistein, Selumetinib, AZD-6244, Vatalanib, P1446A-05, AG-024322, ZD1839, P276-00, GW572016 or a mixture thereof.

In some aspects, the PI3K inhibitor is selected from the group of PI3K inhibitors consisting of buparlisib, idelalisib, BYL-719, dactolisib, PF-05212384, pictilisib, copanlisib, copanlisib dihydrochloride, ZSTK-474, GSK-2636771, duvelisib, GS-9820, PF-04691502, SAR-245408, SAR-245409, sonolisib, Archexin, GDC-0032, GDC-0980, apitolisib, pilaralisib, DLBS 1425, PX-866, voxtalisib, AZD-8186, BGT-226, DS-7423, GDC-0084, GSK-21 26458, INK-1 1 17, SAR-260301, SF-1 1 26, AMG-319, BAY-1082439, CH-51 32799, GSK-2269557, P-71 70, PWT-33597, CAL-263, RG-7603, LY-3023414, RP-5264, RV-1729, taselisib, TGR-1 202, GSK-418, INCB-040093, Panulisib, GSK-105961 5, CNX-1351, AMG-51 1, PQR-309, 17beta-Hydroxywortmannin, AEZS-129, AEZS-136, HM-5016699, IPI-443, ONC-201, PF-4989216, RP-6503, SF-2626, X-339, XL-499, PQR-401, AEZS-132, CZC-24832, KAR-4141, PQR-31 1, PQR-316, RP-5090, VS-5584, X-480, AEZS-126, AS-604850, BAG-956, CAL-130, CZC-24758, ETP-46321, ETP-471 87, GNE-317, GS-548202, HM-032, KAR-1 139, LY-294002, PF-04979064, PI-620, PKI-402, PWT-143, RP-6530, 3-HOI-BA-01, AEZS-134, AS-041 164, AS-252424, AS-605240, AS-605858, AS-606839, BCCA-621 C, CAY-10505, CH-5033855, CH-51 08134, CUDC-908, CZC-1 9945, D-106669, D-87503, DPT-NX7, ETP-46444, ETP-46992, GE-21, GNE-123, GNE-151, GNE-293, GNE-380, GNE-390, GNE-477, GNE-490, GNE-493, GNE-614, HMPL-51 8, HS-104, HS-1 06, HS-1 16, HS-173, HS-196, IC-486068, INK-055, KAR 1 141, KY-1 2420, Wortmannin, Lin-05, NPT-520-34, PF-04691503, PF-06465603, PGNX-01, PGNX-02, PI 620, PI-103, PI-509, PI-516, PI-540, PIK-75, PWT-458, RO-2492, RP-5152, RP-5237, SB-201 5, SB-2312, SB-2343, SHBM-1009, SN 32976, SR-13179, SRX-2523, SRX-2558, SRX-2626, SRX-3636, SRX-5000, TGR-5237, TGX-221, UCB-5857, WAY-266175, WAY-266176, EI-201, AEZS-131, AQX-MN100, KCC-TGX, OXY-1 1 1 A, PI-708, PX-2000, and WJD-008.

It is contemplated that the cancer therapy can comprise an antibody, peptide, polypeptide, small molecule inhibitor, siRNA, miRNA or gene therapy which targets, for example, epidermal growth factor receptor (EGFR, EGFR1, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family (IGF-1R); platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family; AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family; MuSK receptor family; Transforming growth factor alpha (TGF-α), TGF-α receptor; Transforming growth factor-beta (TGF-.beta.), TGF-.beta. receptor; Interleukin 13 receptor alpha2 chain (1L13Ralpha2), Interleukin-6 (IL-6), 1L-6 receptor, Interleukin-4, IL-4 receptor, Cytokine receptors, Class I (hematopoietin family) and Class II (interferon/1L-10 family) receptors, tumor necrosis factor (TNF) family, TNF-α, tumor necrosis factor (TNF) receptor superfamily (TNTRSF), death receptor family, TRAIL-receptor; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), beta-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: beta-Dgalactose 2-alpha-Lfucosyltraosferase (LDLR/FUT) fusion protein, HLA-A2, arginine to isoleucine exchange at residue 170 of the alpha-helix of the alpha2-domain in the HLA-A2 gene (HLA-A*201-R1700, MLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDXS, PTPRK, K-ras (KRAS2), N-ras (NRAS), HRAS, RBAF600, SIRT2, SNRPD1, SYT-SSX1 or-SSX2 fusion protein, Triosephosphate Isomerase, BAGE, BAGE-1, BAGE-2,3,4,5, GAGE-1,2,3,4,5,6,7,8, GnT-V (aberrant N-acetyl giucosaminyl transferase V, MGATS), HERV-K-MEL, KK-LC, LAGE, LAGE-1, CTL-recognized antigen on melanoma (CAMEL), MAGE-A1 (MAGE-1), MAGE-A2, MAGE-A3, MAGE-A4, MAGE-AS, MAGE-A6, MAGE-A8, MAGE-A9, MAGE-A10, MAGE-A11, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gp100, gp100/Pme117 (SILV), tyrosinase (TYR), TRP-1, HAGE, NA-88, NY-ESO-1, NY-ESO-1/LAGE-2, SAGE, Sp17, SSX-1,2,3,4, TRP2-1NT2, carcino-embryonic antigen (CEA), Kallikfein 4, mammaglobm-A, OA1, prostate specific antigen (PSA), prostate specific membrane antigen, TRP-1/gp75, TRP-2, adipophilin, interferon inducible protein absent in nielanorna 2 (AIM-2), BING-4, CPSF, cyclin D1, epithelial cell adhesion molecule (Ep-CAM), EpbA3, fibroblast growth factor-5 (FGF-5), glycoprotein 250 (gp250intestinal carboxyl esterase (iCE), alpha-feto protein (AFP), M-CSF, mdm-2, MUCI, p53 (TP53), PBF, FRAME, PSMA, RAGE-1, RNF43, RU2AS, SOX10, STEAP1, survivin (BIRCS), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SPO11, TPX1, NY-SAR-35, FTHLI7, NXF2 TDRD1, TEX 15, FATE, TPTE, immunoglobulin idiotypes, Bence-Jones protein, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, neuron-specific enoJase, heat shock protein gp96, GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr vims (EBV) proteins (EBV latent membrane proteins-LMP1, LMP2; others), Hepatitis B or C virus proteins, and HIV proteins.

In some embodiments, the cancer therapy comprises the use of checkpoint inhibitor. In some embodiments, the checkpoint inhibitor is a small molecule, an inhibitory nucleic acid, an inhibitory polypeptide, antibody or antigen-binding domain thereof, or antibody reagent. In some embodiments, the checkpoint inhibitor is an antibody or antigen-binding domain thereof, or antibody reagent binds an immune checkpoint polypeptide and inhibits its activity. Common checkpoints that are targeted for therapeutics include, but are not limited to PD-L1, PD-L2, PD-1, CTLA-4, TIM-3, LAG-3, VISTA, and TIGIT. In some embodiments, the checkpoint inhibitor is an antibody or antigen-binding domain thereof, or antibody reagent binds a PD-1, PD-L1, or PD-L2 polypeptide and inhibits its activity.

Inhibitors of known checkpoint regulators (e.g., PD-L1, PD-L2, PD-1, CTLA-4, TIM-3, LAG-3, VISTA, or TIGIT) are known in the art. Non-limiting examples of checkpoint inhibitors (with checkpoint targets and manufacturers noted in parentheses) can include: MGA271 (B7-H3: MacroGenics); ipilimumab (CTLA-4; Bristol Meyers Squibb); pembrolizumab (PD-1; Merck); nivolumab (PD-1; Bristol Meyers Squibb); atezolizumab (PD-L1; Genentech); IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); avelumab (PD-L1; Merck); durvalumab (PD-L1; Medimmune); and TSR-022 (TIM3; Tesaro).

In some embodiments, the checkpoint inhibitor inhibits PD-1. PD-1 inhibitors include, but are not limited to Pembrolizumab (Keytruda™), Nivolumab, AUNP-12, and Pidilizumab. In another embodiment, the checkpoint inhibitor inhibits PD-L1. PD-L1 inhibitors include, but are not limited to Atezolizumab, MPDL3280A, Avelumab, and Durvalumab.

In some embodiments, the cancer therapy comprise surgical intervention to remove cancerous tissue. In some embodiments, the cancer is bladder cancer and the surgical intervention comprises a full cystectomy and/or transurethral resection of bladder tumor (TURBT).

In another aspect, described herein is a method of treating a cancer associated with elevated circulating levels hyaluronidase 2-positive myeloid cells, the method comprising detecting an elevated level of hyaluronidase 2-myeloid cells in a blood sample from the subject as described herein and administering a hyaluronidase 2 inhibitor to the subject. In some embodiments, the hyaluronidase 2 inhibitor is an anti-hyaluronidase 2 antibody or CAR T cells specific for hyaluronidase 2.

It is contemplated that the methods herein reduce tumor burden, and/or reduce metastasis in the subject, and/or reduce or prevent the recurrence of tumors once the cancer has gone into remission. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

Monitoring efficacy of cancer therapy is also contemplated. In this regard, the disclosure provides a method of monitoring efficacy of treatment in a subject undergoing cancer therapy comprising detecting a level of hyaluronidase 2-positive myeloid cells in a blood sample from the subject before and after treatment with a cancer therapy, wherein a decreased level of hyaluronidase 2-positive myeloid cells identified in the blood sample after cancer therapy compared to before treatment began is indicative of effective treatment. A decreased level of hyaluronidase 2-positive myeloid cells (such that the level of hyaluronidase 2-positive myeloid cells in the sample is commensurate with a level of hyaluronidase 2-positive myeloid cells in healthy subjects) in the sample is indicative of effective cancer therapy.

All of the references cited herein, including patents, patent applications, literature publications, and the like, are hereby incorporated in their entireties by reference.

### EXAMPLES

### Materials and Methods

Human subjects: Freshly excised bladder tumor tissue and peripheral blood from 24 patients diagnosed with urothelial carcinoma of the bladder were collected during cystectomy or transurethral resection (TURBT). Samples of normal bladder tissue were collected from patients undergoing cystectomy.

Reagents and culture medium: The proteinase K and Benzonase were purchased from Sigma-Aldrich. Commercial polydisperse hyaluronan samples HAIM (MW 750-1000 kDa), 700K (500-749 kDa), 500K (301-450 kDa), 200K (151-300 kDa), 10K (10-20 kDa) and 5K (<10 kDa) were obtained from Lifecore Biomedical (Chaska, MN). Biotinylated hyaluronan-binding protein (HABP) was supplied by Millipore-Sigma. Streptavidin conjugated with PE or FITC was purchased from Biolegend (San Diego, CA). Hyaluronidase 2 polyclonal antibody conjugated with PE or Alexa-488 was obtained from Bioss Antibodies. All other antibodies used for immune fluorescence and flow cytometric analysis were acquired from Biolegend. In vitro experiments were conducted using complete culture media consisting of RPMI 1640 medium supplemented with 20 mM HEPES, 200 U/ml penicillin, 50 μg/ml streptomycin (all from Hyclone) and 10% FBS from ATCC (Manassas, VA).

Preparation of tissue slices from human normal and bladder cancer tissues: The precision-cut tissue slices, 2-4 mm in diameter and 100-300-micron thick, were produced from cancer and normal bladder tissues using a Compresstome vibratome VF-300-0Z. After cutting, tissue slices were placed into 24-well cell culture plates in complete RPMI-1640 medium supplemented with 10% FBS and antibiotics and cultured at 37° C. in a humidified CO₂ incubator. Cell viability of cultured tissue slices was tested using the Live/Dead cell viability kit purchased from Invitrogen.

Isolation of CD11b myeloid cells from peripheral blood of cancer patients: PBMCs were isolated from bladder cancer patients by gradient density centrifugation using Lymphoprep (Accu-Prep, 1.077 g/ml, Oslo, Norway). CD11b myeloid cells were purified from PBMCs by positive selection using the anti-CD11b microbeads and columns (Miltenyi Biotec). Briefly, cells were incubated with beads conjugated with anti-mouse CD11b and positively selected on LS columns. Viability of all recovered cells was 95%, as determined by trypan blue exclusion.

Human cancer cell lines: The human T24 bladder cancer cell line was purchased from the ATCC (Manassas, VA). Tumor cells were maintained at 37° C. in a 5% CO₂ humidified atmosphere in complete culture medium.

Preparation of tumor-conditioned medium: The source of tumor-conditioned medium (TCM) was bladder cancer tissue slice cultures or cultured T24 cell line. To prepare TCM, conditioned medium was collected 2-3 days after tissue or cell culture initiation, centrifuged, aliquoted and stored at −80° C.

Cytokine and chemokine profiling: Human bladder cancer and normal tissue slices were cultured in a humidified CO₂ incubator at 37° C. For profiling of cytokines in tissue conditioned media, cell-free culture supernatants were collected and stored at −80° C. Presence of 105 proteins in supernatants was evaluated using human cytokine and chemokine XL proteome array kit from R&D Systems (Minneapolis, MN).

Visualization of tumor-produced HA: Cancer or normal bladder tissue slices were cultured for 7-14 days in 24-well cell culture plates in a humidified CO₂ incubator at 37° C. to allow for production of HA. At the end of incubation, tissue-produced HA was found settled at the bottom of the culture plate wells. To monitor and visualize accumulation of tissue-produced HA fragments on the plastic surface, the tissue slices and culture medium were removed at different time points. The empty wells were washed with warm PBS and fixed with 4% formaldehyde for 30 min. After fixation, plate wells were washed with PBS containing 2% FBS and incubated overnight with biotinylated HA-binding protein (3 μg/ml, Calbiochem-EMD Millipore) at 4° C. (16). Next day, after washing the wells with PBS containing 2% FBS, streptavidin conjugated with fluorochrome was added to the wells and incubated for 30 min at 4° C. Plates were then washed with PBS and the bottoms of the wells were visualized using EVOS (Invitrogen) or Lionheart (Biotek Instruments) immunofluorescent imaging microscopes.

Evaluation of HA size: HA size analysis was determined using polyacrylamide gel electrophoresis as described previously (17). Briefly, conditioned medium from cancer and normal bladder cancer tissue slices was centrifuged, aliquoted and stored at −80° C. To prepare samples for HA size analysis, thawed samples were digested with proteinase K to remove proteins, benzonase for depletion of nucleic acids (RNA, DNA) and ethanol to extract lipids was added. Samples along with HA standards were then subjected for polyacrylamide electrophoresis. The tissue-produced HA was visualized on the gel by staining with “Stains All” (Sigma-Aldrich) dye.

Immunofluorescent microscopy and flow cytometry: Immunofluorescent staining and flow cytometry analysis was performed as described previously (18, 19). Image analysis was done using Gen 5 Prime v 3.08 software (Biotek Instruments). Flow cytometry data and microscope pictures shown are representative of at least two separate determinations.

Statistical analysis: The statistical significance between values was determined by the Student t test. All data was expressed as the mean +SD. Probability values ≥0.05 were considered non-significant.

### Example 1—Enhanced HA Degradation in Bladder Cancer Tissue Results in Accumulation of LMW-HA Fragments

Normal bladder tissues produced predominantly long, structured linear pericellular hyaluronan (HA), whereas the bladder cancer samples generated highly fragmented tissue-associated HA (data not shown).

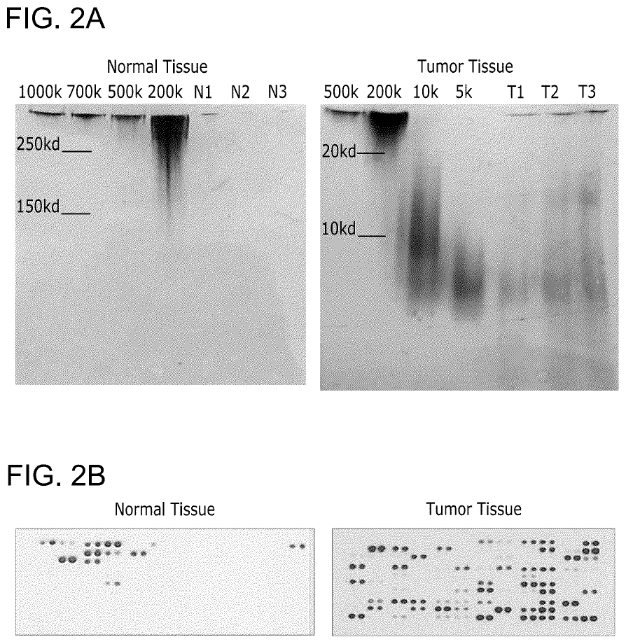

The size of HA secreted by normal and bladder cancer tissues was further characterized by gel electrophoresis. Interestingly, slices from normal bladder tissues (FIG. 2A, left panel) produced mostly HMW-HA (>200 kDa) with undetectable levels of LMW-HA. In contrast, human bladder cancer tissues generated fragmented HA with a prevalence of LMW-HA with MW <20 kDa (FIG. 2A, right panel) confirming the prior observation of increased HA fragmentation in bladder cancer tissues.

### Example 2—Enhanced HA Degradation in Tumor Tissue is Associated with Elevated Secretion of Inflammatory, Angiogenic and Tumor-Supporting Factors

Accumulating evidence suggests that HA fragments with low molecular weight are able directly promote tumor progression by stimulating secretion of various factors that enhance tumor migration, invasion, inflammation, and angiogenesis. (5, 6, 9). To assess and compare the cytokine profile produced by normal and bladder cancer tissues, the tumor conditioned medium (TCM) collected from normal and bladder cancer tissue slices was analyzed for the presence of 105 cytokines and chemokines using proteome multiplex assay. As shown in FIG. 2B (left panel), normal bladder tissue secreted detectable levels of nine proteins: adiponectin, ApoA1, chitinase3-like1, compliment factor D, C-reactive protein, endoglin, CXCL5, IL-8, and angiogenin. In TCM obtained from bladder cancer tissue, 34biologically active factors were detected (FIG. 2B, right panel). Among them, there were several angiogenic factors: VEGF, angiopoetin-2, angiogenin, and trombospondin-1; multiple chemokines and growth factors associated with inflammation and recruitment of different cell subsets to the tumor site such as CCL2, CCL7, CXCL1, CXCL5, CCL20, G-CSF, GM-CSF, lipocalin. The bladder cancer tissue also produced proteins involved in immune regulation (IL-6, IL-8, IL-11, IL-18Bpa, IL-24, IL-1R4, osteopontin) and tissue remodeling (MMP9, chitinase3-like1, dipeptyl-peptidase IV, IGFBP-2, uPAR, trombospondin). Proteins implicated in tumor cell invasion and migration, such as DKK1, cripto-1, HGF, were also detected. Taken together, this data suggests that accumulation of LMW-HA fragments within bladder cancer is accompanied by elevated production of multiple bioactive factors that are associated with tumor growth and progression.

### Example 3—Detection of Hyal2-Expressing Tumor-Associated Myeloid Cells in Human Bladder Cancer

Increased levels of inflammatory chemokines and cytokines produced by the tumor facilitate cancer-associated inflammation and drive recruitment of myeloid cells to the tumor microenvironment where they become involved in bidirectional crosstalk with tumor cells (20, 21). It was oberserved that bladder cancer tissue was infiltrated with myeloid cell subsets when compared to normal bladder tissue (data not shown). Moreover, the tumor-infiltrating cells frequently associated with enhanced HA fragmentation (data not shown). It was hypothesized that the enhanced HA degradation within bladder cancer is attributed to the presence and activity of tumor-recruited myeloid cells. To test this hypothesis, HA produced by bladder cancer tissue slices was co-stained with common myeloid cell marker CD11b. It was observed that the CD11b+ cells localized in close proximity and some adhered to highly degraded HA fragments (data not shown).

Cancer tissue samples were stained for HA, Hyal2 and CD11b. Results showed the presence of Hyal2-expressing cells in tumor-bladder tissue slice cultures among both adherent and non-adherent cells. Moreover, Hyal2 expression was observed in areas with highly fragmented HA and associated with a subset of tumor-infiltrating CD11b myeloid cells (data not shown), suggesting a possible contribution of these cells to the enhanced HA degradation in bladder cancer tissue.

### Example 4—Hyal2-Expressing Myeloid Cells in Peripheral Blood of Cancer Patients

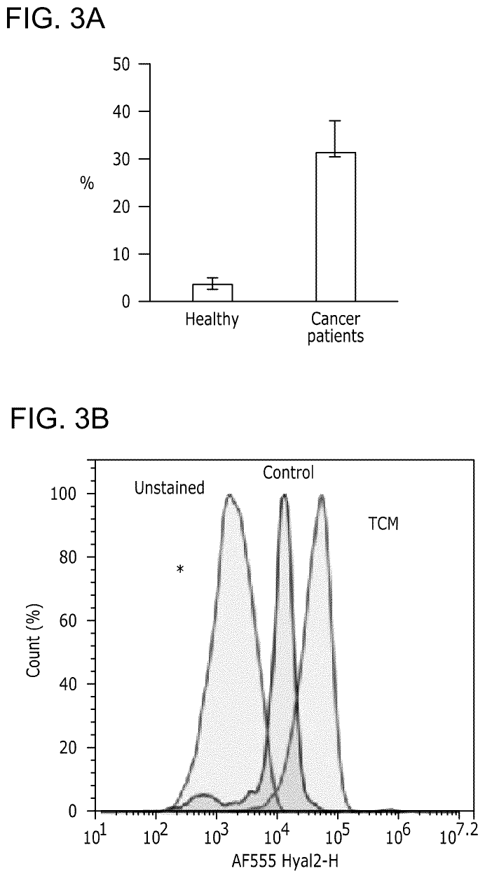

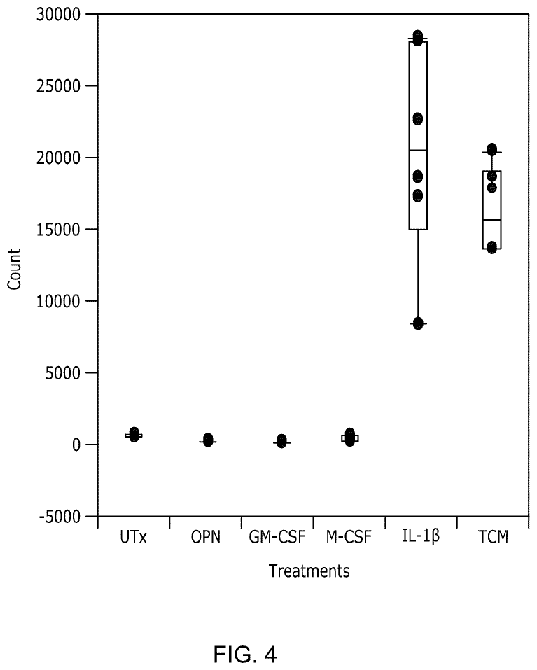

The peripheral blood of bladder cancer patients contain increased numbers of myeloid cells, including granulocytic and monocytic myeloid-derived suppressor cells (MDSCs) as compared to healthy individuals (22). To explore whether Hyal2-expressing myeloid cells could be also detected in peripheral blood, the CD11b⁺ cell populations was isolated from peripheral blood of bladder cancer patients and healthy donors, and stained with anti-Hyal2 antibodies. As shown in FIG. 4, myeloid cells from blood of cancer patients contain significantly more Hyal2-positive cells as compared to healthy individuals. It was also found that exposure of myeloid cells to TCM further stimulated expression of Hyal2 (FIG. 3B), suggesting that expression of this enzyme could be up-regulated in myeloid cells upon recruitment to the tumor microenvironment with abundance of tumor-derived factors. Furthermore, a significant portion of Hyal2⁺ myeloid cells also co-expressed the antigen-presenting cell marker HLA-DR. See FIG. 4C showing 13.7% in non-stimulated vs 46.7% in TCM-stimulated CD11b myeloid cells. Interestingly, in both TCM-treated and non-treated myeloid cells expression of Hyal2 was detected in dispersed granular form with both intracellular and cell surface localization (data not shown). However, engagement of cells with antibodies against CD44 promoted significant changes in both cellular shape and Hyal2 localization, supporting the idea that the CD44 receptor is involved in the regulation of Hyal2 function (12, 13, 23, 24).

### Example 5—IL-1beta Stimulates HA-Degrading Activity of Hyal2+ Myeloid Cells

To examine whether TCM-stimulated CD11b⁺Hyal2⁺HLA-DR⁺ cells are functionally active, HA-degrading activity was characterized. To this end, the CD11b cells isolated from peripheral blood of cancer patients were added to the culture plates pre-coated with commercial HA (MW 200 kDa), and cultured in the presence of TCM for 10 days. Visualization of HA was executed using biotinylated hyaluronan-binding protein (HABP). It was observed that TCM-stimulated myeloid cells were able to promote degradation of extracellular HA. Furthermore, by end of the culture (10 days), most of the TCM-stimulated myeloid cells acquired the shape of mature antigen-presenting cells and were able to internalize the fragmented HA (data not shown).

Next, the potential cytokines/factors that could be involved in stimulation of HA-degrading activity mediated by Hyal2-expressing myeloid cells was examined. Hyal2⁺ cells were isolated from peripheral blood of cancer patients and cultured in the presence or absence of following: human recombinant GM-CSF, M-CSF, IL-1beta, osteopontin or TCM. HA-degrading activity of cultured Hyal2⁺ cells was evaluated by the visualization of degraded HA using IF microscopy, and quantification of small HA fragments detected on plastic surface in cell culture plates using imaging software. Obtained data clearly demonstrated that only IL-1beta among tested cytokines has been able to promote strong HA-degrading activity in Hyal2⁺ myeloid cells (FIG. 4).

### Example 6—Bone Marrow as a Source of Hyal2-Expressing Cells

Further analysis of myeloid cells obtained from peripheral blood of bladder cancer patients revealed that Hyal2-positive cells did not express the monocytic marker CD14 (data not shown). Long-term culture of Hyal2⁺ cells showed that these cells retained the antigen-presenting cell marker HLA-DR, but expression of Hyal2 becomes somewhat weaker and its localization was more intracellular overtime (data not shown). Importantly, the HLA-DR⁺CD11b⁺ cells and Hyal2⁺HLA-DR⁺ myeloid cells can be detected in bladder cancer tissue (FIG. 6C and FIG. 6D, respectively) in close proximity to areas that enriched for highly fragmented HA (FIG. 6D, right image), suggesting the contribution of this myeloid cell subset to the process of HA degradation in the tumor microenvironment. Finally, taking into consideration that the primary location in the body where myelopoiesis takes place is bone marrow, it is hypothesized that bone marrow may potentially serve as a source of Hyal2⁺ myeloid cells. Analysis of CD11b myeloid cells isolated from normal human bone marrow, confirmed a significant presence of Hyal2-expressing myeloid cells in bone marrow (FIG. 6E). Together, these data demonstrate that Hyal2-expressing myeloid cells can be detected in bladder cancer tissue, in close proximity to the HA degradation points.

### REFERENCES

Girish, et al., Life Sciences. 80:1921-1943, 2007.
Toole, B.P., Nat. Rev. Cancer. 4:528-539, 2004.
Sironen, et al., Exp. Cell Res., 317:393-91, 2011.
Simpson et al., 13:5664-5680, 2008.
Schmaus et al., Cancer Metastasis Rev, 33:1059-1079, 2014.
Turley et al., Cancer Res, 76 (9): 2507-2512, 2016.
Tian X et al. Nature, (499): 346-349, 2013.
Ooki Tet al., Dev. Cell, 49 (4): 590-604, 2019.
Sokolowska, et al., J. Biol. Chem. 289:4470-4488, 2014.
Kramer et al., Eur. Urol., 57:86-94, 2010.
Van Der Heijden AG et al., Eur J Cancer, 64:127-36, 2016.
Stern et al., Semin Cancer Biol, 18:275-280, 2008.
Harada et al., J. Biol. Chem., 282 (8): 5597-5607, 2007.
Eruslanov et al., International Journal of Cancer, 130 (5): 1109-19, 2012.
Puttman K. et al, Bladder Cancer, 5 (2): 103-11, 2019.
Petrey, et al., J Biol. Chem. 291:24324-24334, 2016.
Bhilocha et al., Anal. Biochem, 417 (1): 41-49, 2011.
Daurkin et al., Cancer Research, 71 (20): 6400-9, 2011.
Prima, et al., PNAS, (114): 1117-1122, 2007.
Biswas et al., Nat. Immunol., 11:889-896, 2010.
Gabrilovich, et al., Nat. Rev. Immunol., 12:253-268, 2012.
Crispen PL and Kusmartsev S. Mechanisms of immune evasion in bladder cancer. Cancer Immunology and Immunotherapy, 2019, doi: 10.1007/s00262-019-02443-4.
Bourguignon et al., J Biol Chem., 279:26991-27007, 2004.
Duterte et al., J Biol Chem., 284:33495-33508, 2009.
Jiang, Physiological Reviews, 91(1), 221-264, 2011.
Cnanmee et al., Cancer Lett, 375(1): 20-30, 2016.
Jiang, et al., Nat Med, 11(11), 1173-1179, 2015.
Voelcker, et al., Exp. Dermatol., 17(2), 100-107, 2008.
West et al., Science, 228(4705): 1324-6, 1985.
Matou-Nasri, et al., International Journal of Oncology, 35(4), 761-773, 2009.
Gao, et al., Matrix Biology: Journal of the International Society for Matrix Biology, 29(2): 107-116, 2010.
Itano, et al., Cancer Res, 59(10): 2499-2504, 1999.
Schmaus, et al., Br J of Cancer, 2014.332, 2014.
Rai et al., Proc Natl Acad Sci USA., 98:4443-4448, 2001.
Andre et al., Biochem Biophys Res Commun., 411:175-179, 2011.
Stern R. Glycobiology, 105-115, 2003.
Czoka A.b et al. The six hyaluronidase-like genes in the human and mouse genomes. Matrix Biol, 2001 20:499-508.
Stern R., Pathol Biol (Paris), 53(7): 372-82, 2005.
Yoshida M et al, Arthritis Res. Ther., 6:514-20, 2004.
Tanimoto k et al., Ann Biomed. Eng., 38:1618-25, 2010.
Jang et al., Cancer Res, 80(6): 1342-56, 2020.
Kaplanov et al., Proc Natl Acad Sci USA., 116(4): 1361-1369, 2019

1. A method of diagnosing cancer in a subject comprising
contacting a blood sample from the subject with a hyaluronidase 2-binding agent and
detecting a level of hyaluronidase 2-positive myeloid cells in the blood sample, wherein an elevated level of hyaluronidase 2-positive myeloid cells in the blood sample identifies the subject as suffering from cancer.

2-11. (canceled)

12. A method of treating cancer in a subject in need thereof, comprising
detecting an elevated level of hyaluronidase 2-myeloid cells in a blood sample from the subject and
administering a cancer therapy to the subject.

13. The method of claim 12, wherein the detection step comprises contacting the blood sample from the subject with a hyaluronidase 2 binding agent.

14. The method of claim 13, wherein the hyaluronidase 2 binding agent is an anti-hyaluronidase 2 antibody.

15. The method of claim 14, wherein the antibody comprises a set of 6 CDRs set forth in SEQ ID NOs: 6-11.

16. The method of claim 14, wherein the antibody comprises a light chain variable region set forth in SEQ ID NO: 2.

17. The method of claim 14, wherein the antibody comprises a heavy chain variable region set forth in SEQ ID NO: 4.

18. The method of claim 12, wherein the cancer therapy comprises radiotherapy, chemotherapy or immunotherapy.

19. (canceled)

20. An antibody-drug conjugate comprising an hyaluronidase 2 (Hyal2) antibody conjugated to a cytotoxic agent.

21. The antibody-drug conjugate of claim 20, wherein the cytotoxic agent is a dihydrofolate reductase inhibitor, a thymidylate synthase inhibitor, a DNA intercalator, a DNA cleaver, a topoisomerase inhibitor, anthracycline, vinca, mitomycin, bleomycin, a cytotoxic nucleoside, a pteridine, a diynene, a podophyllotoxin, a dolastatin, a maytansinoid, a differentiation inducer, and a taxol.

22. The antibody-drug conjugate of claim 20, wherein the cytotoxic agent is a chemotherapeutic agent.

23. The antibody-drug conjugate of claim 22, wherein the chemotherapeutic agent is methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin and podophyllotoxin derivatives such as etoposide or etoposide phosphate, vinblastine, vincristine, vindesine, taxanes including taxol, taxotere retinoic acid, butyric acid, N8-acetyl spermidine, camptothecin, calicheamicin, esperamicin, ene-diynes, duocarmycin A, duocarmycin SA, calicheamicin, camptothecin, maytansinoids (including DM1), monomethylauristatin E (MMAE), monomethylauristatin F (MMAF), or maytansinoids (DM4).

24. The antibody-drug conjugate of claim 20, wherein the antibody is conjugated with Doxorubicin, Docetaxel (Taxotere), Cisplatin, Gemcitabine, Carboplatin, Etoposide, Cyclophosphamide, Epirubicin, Vincristine, Methotrexate, 5-fluoroucil, or Mitomycin C.

25. The antibody-drug conjugate of claim 20, wherein the antibody comprises a set of 6 CDRs set forth in SEQ ID NOs: 6-11.

26. The antibody-drug conjugate of claim 20, wherein the antibody comprises a light chain variable region set forth in SEQ ID NO: 2.

27. The antibody-drug conjugate of claim 20, wherein the antibody comprises a heavy chain variable region set forth in SEQ ID NO: 4.

28. A method of treating cancer in a subject in need thereof, comprising administering the antibody-drug conjugate of claim 20 to the subject.

29. The method of claim 28, wherein the cancer is prostate cancer, bladder cancer, breast cancer, brain cancer or lung cancer.
